(19) United States
(12) Patent Application Publication
(10) Pub. No.: US 2021/0137391 A1
(43) Pub. Date: May 13, 2021

(54) HYBRID SENSING BASED PHYSIOLOGICAL MONITORING AND ANALYZING METHOD AND SYSTEM

(71) Applicant: ANIWEAR COMPANY LIMITED, Hong Kong (CN)

(72) Inventor: Nin Fung Joseph MAK, Hong Kong (CN)

(21) Appl. No.: 16/963,422

(22) PCT Filed: Jan. 17, 2019
(86) PCT No.: PCT/IB2019/050371

§ 371 Date: Jul. 20, 2020

(30) Foreign Application Priority Data
Jan. 19, 2018 (CN) 201810053929.9

(51) Int. Cl. A61B 5/0205; A61B 5/00

(52) U.S. Cl. CPC A61B 5/0205; A61B 5/7203; A61B 5/7257; A61B 2562/0219; A61B 5/7225; A61B 5/7275; A61B 2562/0247; A61B 5/7264

(57) ABSTRACT

The invention relates to a physiological detection and analysis method based on hybrid sensing, construct algorithm statistical model through experiment, then input the collected physiological information data of the target creature into the algorithm statistical model after performing noise reduction processing, then obtain the output target, said output target is used as the analysis report, or compare with the database to obtain the analysis report, then judge the health state of the target creature; further provides a physiological detection and analysis system based on hybrid sensing, which comprises sensors that collect data, data recording unit, data analysis unit, and report receiving unit; the invention realizes the comprehensive analysis for target creature through collecting various aspects of physiological data of the target creature, makes the analysis result more accurate, reliable, convenient and faster, and improves the efficiency of physiological detection and disease detection.

### TECHNICAL FIELD

The invention relates to disease diagnosis technology, in particular to a physiological detection and analysis method and system based on hybrid sensing.

### BACKGROUND OF THE INVENTION

The judgment of the disease or healthy state in the prior art is generally judged by a testing machine. However, the accuracy of the testing result is not high due to the interference of external factors and the incomplete physiological data obtained due to conditional restrictions, which may easily cause misdiagnosis.

### SUMMARY OF THE INVENTION

### Technical Problem

In view of the defects or deficiencies in the prior art, the technical problem to be solved by the invention is to provide a technical solution that can solve the misdiagnosis in the judgement of the healthy state.

### Solution to the Problem

In order to realize the above purpose, the technical solution adopted by the invention is to provide a hybrid sensing-based physiological monitoring and analyzing system, which comprising the following steps:

S1. Construct algorithm statistical model through experiment;

S2. Collect physiological information data of the target creature; wherein said physiological information data comprises electrical physiological information, mechanical physiological information and body movement data of the target creature; by collecting electrical physiological information, mechanical physiological information and body movement data of the target creature to ensure the comprehensiveness of the information;

S3. Perform noise reduction processing on said physiological information data through signal processing method, and extract time-domain features and/or frequency-domain features of different physiological information data through feature extraction method; wherein, said feature extraction method is one or the combination of Fourier Transform, frequency band power calculation, time frequency analysis, wavelet decomposition or waveform detection, or one or the combination of other feature extraction method(s) which extracting time-domain features or frequency-domain features of physiological information data; improve the signal-to-noise ratio of physiological information data, excluding distortion or abnormal information data caused by external interference or other uncontrollable factors;

S4. Input the time-domain features and/or frequency-domain features extracted from electrical physiological information, mechanical physiological information and body movement data into the algorithm statistical model for calculation, to obtain the output target; wherein, said algorithm statistical model comprises algorithm statistical model of heart rate check, algorithm statistical model of blood pressure check, algorithm statistical model of heart rate variability check, algorithm statistical model of respiration rate check, algorithm statistical model of emotion check, algorithm statistical model of cardiac output check and algorithm statistical model of body movement check. The said output target comprises heart rate analysis, blood pressure analysis, heart rate variation analysis, respiration rate analysis, emotion analysis, cardiac output analysis and body movement analysis corresponding to said algorithm statistical model, and may also comprise algorithm statistics of other physiological information models;

S5. The said output target is used as the analysis report and reported back to the report receiving unit, or said output target is compared with the past database to obtain the analysis report and reported back to the report receiving unit, wherein the said past database comprises: the past physiological information data of said target creature, and the past physiological information data group of the creature of the same or different races or breeds as said target creature. The past database generally stores the physiological information data of the target creature, or creature of the same or similar races, families, categories, ages, and sizes as the target creature. By comparing the output target with the data, the analysis report of target creature is obtained, then the data analysis unit sends the analysis report to the report receiving unit, so that the professionals can make recommendations based on the report.

As the further improvement of the invention, said S1 further comprises the following steps:

S11. Collect the experimental physiological information data of experimental object through the sensor;

S12. Improve signal-to-noise ratio of experimental physiological information data through the signal processing method;

S13. Extract time-domain features and/or frequency-domain features of different experimental physiological information data through the feature extraction method, wherein said feature extraction method is: Fourier Transform, frequency band power calculation, time frequency analysis, wavelet decomposition and waveform detection;

S14. The statistical model is constructed by inputting the time-domain features and/or frequency-domain features of the experimental physiological information data into the machine learning system, and train the statistical model to obtain algorithm statistical model.

As the further improvement of the invention, said S14 further comprises the following steps:

S141. Standard statistical testing parameters and the acceptable deviation degree of algorithm results are preset in said machine learning system;

S142. Said machine learning system extracts the subset of the relevant time-domain features and/or frequency-domain features of said experimental physiological information data through the feature extraction method to construct different combinations of models, and compare the calculation result of the statistical model with the physiological result obtained by the standard measurement method, check whether it meets the preset statistical testing parameter and the acceptable result deviation degree;

S143. If it does not meet, then remove the time-domain features and/or frequency-domain features of the test from said statistical model;

S144. Construct the algorithm statistical model by selecting the feature subset with the highest accuracy and statistical parameter values.

As the further improvement of the invention, said electrical physiological information comprises electrocardiogram and electrical respiration measurement diagram.

As the further improvement of the invention, said mechanical physiological information comprises seismocardiogram, ballistocardiogram and mechanical respiration measurement diagram.

As the further improvement of the invention, said output target comprises body movement, respiration rate, heart rate, heart rate variability, blood pressure, emotion and cardiac output.

The invention further provides a physiological detection and analysis system based on hybrid sensing, which comprises some sensors, data recording unit, data analysis unit, and report receiving unit; said data analysis unit: used for analyzing the physiological information data of the target creature collected by said sensor after being processed by said data recording unit, and sending the analysis report to said report receiving unit.

As the further improvement of the invention, said sensor comprises electrocardiography sensor, accelerometer, motion sensor and pressure sensor; said data recording unit comprises the central processor for measuring, recording or storing physiological information data collected by said sensor, said central processor is also used for sending said physiological information data to said data analysis unit.

As the further improvement of the invention, said data analysis unit comprises past database, real-time database and analysis platform that can construct and train algorithm statistical models through machine learning method;

Said past database comprises: the past physiological information data of said target creature, and the past physiological information data group of the creature of the same or different races or breeds as said target creature;

Said real-time database comprises the physiological information data of said target creature.

As the further improvement of the invention, said data analysis platform is also used for: improving the signal-to-noise ratio of said physiological information data, extracting time-domain features and/or frequency-domain features of different physiological information data through feature extraction method.

### Advantageous Effect of the Invention

The invention realizes the comprehensive analysis of physiological information for target creature through collecting various aspects of physiological data of the target creature, makes the analysis result more accurate, reliable, convenient and faster, and improves the efficiency of physiological detection and disease detection.

### BRIEF DESCRIPTION OF THE DRAWINGS

FIG. 1

FIG. 1 is the detection flow chart of the invention;

FIG. 2

FIG. 2 is the system chart of the invention;

FIG. 3

FIG. 3 is the time-domain data image extracted from electrical physiological signal provided by the invention;

FIG. 4

FIG. 4 is the frequency-domain data image extracted from electrical physiological signal provided by the invention;

FIG. 5

FIG. 5 is the time-domain data image extracted from mechanical physiological signal provided by the invention;

FIG. 6

FIG. 6 is the frequency-domain data image extracted from mechanical physiological signal provided by the invention;

FIG. 7

FIG. 7 is the inter-relationship between time-domain features of electrical and mechanical physiological signal provided by the invention;

FIG. 8

FIG. 8 is the data image of the mental state principal component analysis between the time-domain/frequency-domain features extracted from electrical physiological signal and mechanical physiological signal provided by the invention;

FIG. 9

FIG. 9 is the correlation data image between the time-domain features extracted from electrical physiological signal and mechanical physiological signal provided by the invention for analyzing the cardiac output and related parameter image;

FIG. 10

FIG. 10 is a side view of the data collection structure of the analysis system provided by the invention;

FIG. 11

FIG. 11 is the folding or unfolding process diagram of the data collection structure of the analysis system provided by the invention;

FIG. 12

FIG. 12 is the embodiment of collecting target creature data provided by the invention;

Wherein, the numbers represent: 11-Electrocardiography sensor, 12-Accelerometer, 13-Pressure sensor, 2-Data storing unit, 3-Data analysis unit, 4-Report receiving unit and 51-Motion sensor.

### Embodiment

Below further describe the invention combining with drawings and embodiments.

As shown in FIG. 1, the invention provides a physiological detection and analysis method based on hybrid sensing, which comprising the following steps:

S1. Construct algorithm statistical model through experiment;

Specifically, said S1 further comprises the following steps:

S11. Collect the experimental physiological information data of experimental object through the sensor;

Collect a large amount of data during the experiment (human or/and animal, same and different races/breeds, healthy and unhealthy): data collected from sensor (electrical physiological signal, mechanical physiological signal or body movement data), and record the data of each output target at that time, such as body movement, respiration rate, heart rate, heart rate variability, blood pressure, emotion, cardiac output and related parameters, such as cardiac blood output, cardiac ejection fraction, etc. Comprehensive data collection is conducive to the construction of comprehensive algorithm statistical model, it makes the structure more accurate when analyzing the physiological state of target creature in the subsequent applications.

S12. Improve signal-to-noise ratio of experimental physiological information data through the signal processing method; in general, there will inevitably be interference data when collecting data, these interference data will disturb the analysis result and even cause misdiagnosis. The data collected from the sensor, such as electrical physiological signal and mechanical physiological signal, are required to improve the signal-to-noise ratio through correspondingly appropriate signal processing methods before inputting into the machine learning system.

S13. Extract time-domain features and/or frequency-domain features of different experimental physiological information data through the feature extraction method, wherein said feature extraction method is one or the combination of Fourier Transform, frequency band power calculation, time frequency analysis, wavelet decomposition, waveform detection and etc; perform feature extraction of different experimental physiological information data through one or more of said feature extraction methods; The time-domain features and/or frequency-domain features of the extracted experimental physiological information data are generally representative, different time-domain and/or frequency-domain features. The signal processing methods applied for feature extraction comprise but is not limited to Fourier Transform, frequency band power calculation, time frequency analysis, wavelet decomposition, waveform detection (amplitude change and time position) and etc, the system will further extract related information automatically.

The statistical model is constructed by inputting the time-domain features and/or frequency-domain features of the experimental physiological information data into the machine learning system, and train the statistical model to obtain algorithm statistical model.

Specifically, said S14 further comprises the following steps:

S141. Standard statistical testing parameters and the acceptable deviation degree of algorithm results are preset in said machine learning system; for example, the standard statistical testing parameter is preset to be larger than 95% (i.e. p-value <0.05), the value of this significance level depends on the object of the statistical study, and the acceptable deviation of the blood pressure is set to <1 mmHg.

S142. Said machine learning system extracts the subset of the relevant time-domain features and/or frequency-domain features of said experimental physiological information data through the feature extraction method to construct different combinations of models, and compare the calculation result of the statistical model with the physiological result obtained by the standard measurement method, each calculation algorithm is trained separately, and probably have different preset values and parameters; check whether it meets the preset statistical testing parameter and the acceptable result deviation degree; After the statistical model is constructed, the statistical model needs to be trained to become more representative, wherein the relevant machine learning system will use the “feature extraction” calculation method to exclude the insufficiently influential time-domain/frequency-domain features, “feature extraction” will extract and use the relevant data, calculate the required target value and compare it with the experimental data, and check whether it meets the significant level and requirement of prediction error.

S143. If it does not meet, then remove the time-domain features and/or frequency-domain features of the test from said statistical model;

By performing calculations on all data in a loop, until a statistical model that can comprehensively meets the preset level and requirement of prediction error for all data is generated; it should be noted that different output target data has different algorithms, the algorithm statistical model will be composed of different time-domain/frequency-domain features, and has different parameters.

S144. Construct the algorithm statistical model by selecting the feature subset with the highest accuracy and statistical parameter values.

According to the need, the physiological data of the human or animal collected in the experiment can be body movement, respiration rate, heart rate, heart rate variability, blood pressure, emotion, cardiac output, etc.

The Steps after Model is Constructed:

S2. Collect physiological information data of the target creature; wherein said physiological information data comprises electrical physiological information, mechanical physiological information and body movement data of the target creature; by collecting electrical physiological information, mechanical physiological information and body movement data of the target creature to ensure the comprehensiveness of the information.

S3. Perform noise reduction processing on said physiological information data through signal processing method, and extract time-domain features and/or frequency-domain features of different physiological information data through feature extraction method; wherein, said feature extraction method is Fourier Transform, frequency band power calculation, time frequency analysis, wavelet decomposition or waveform detection; improve the signal-to-noise ratio of physiological information data, excluding distortion or abnormal information data caused by external interference or other uncontrollable factors.

S4. Input the time-domain features and/or frequency-domain features extracted from electrical physiological information, mechanical physiological information and body movement data into the algorithm statistical model for calculation, to obtain the output target; wherein, said algorithm statistical model comprises algorithm statistical model of heart rate check, algorithm statistical model of blood pressure check, algorithm statistical model of heart rate variability check, etc., through different physiological information data established in the experiment to establish an algorithm statistical model corresponding to the physiological information data, input the collected physiological information data of the target creature into the corresponding algorithm statistical model for comparison, calculation and analysis, to obtain the corresponding output target; said output target comprises heart rate analysis, blood pressure analysis, heart rate variation analysis and etc corresponding to said algorithm statistical model.

For example, the time-domain features and/or frequency-domain features extracted from electrical physiological information, mechanical physiological information, and body movement data are input into the algorithm statistical model established for heart rate check, only when machine learning is selected, the features related to heart rate will be selected, and the output of the statistical model is the output target of heart rate, which is analyzed according to output target of several physiological information data of the target creature.

S5. The said output target is used as the analysis report and reported back to the report receiving unit 4, or said output target is compared with the past database to obtain the analysis report and reported back to the report receiving unit 4, wherein the said past database comprises: the past physiological information data of said target creature, and the past physiological information data group of the creature of the same or different races or breeds as said target creature. The past database generally stores the physiological information data of the target creature, or creature of the same or similar races, families, categories, ages, and sizes as the target creature. By comparing the output target with the data, the analysis report of target creature is obtained, then the data analysis unit 3 sends the analysis report to the report receiving unit 4, so that the professionals can make recommendations based on the report.

As shown in FIG. 2, the invention further provides a physiological detection and analysis system based on hybrid sensing, which comprises some sensors, data recording unit 2, data analysis unit 3, and report receiving unit 4; said data analysis unit: used for analyzing the physiological information data of the target creature collected by said sensor after being processed by said data recording unit 2, and sending the analysis report to said report receiving unit 4. Wherein, said sensors comprise but is not limited to: the electrocardiography sensor 11, accelerometer 12, motion sensor and pressure sensor 13 used for collecting related information of electrical physiological activity, mechanical physiological activity and body movement; the invention records the electrical physiological and mechanical activities of the cardiovascular system in the manner of synchronization and time locking, and simultaneously measures cardiopulmonary activities and body movement.

As shown in FIG. 3-9, The system can collect real-time electrical physiological information from animal and human through the above sensors. Electrical physiological information comprises, but is not limited to: electrocardiogram (ECG) and electrical respiration measurement; collect real-time mechanical physiological information from animal and human, mechanical physiological information comprises, but is not limited to: seismocardiogram (SCG), ballistocardiogram (BCG) and mechanical respiration measurement; collect real-time body movement data. And extract the time-domain features and frequency-domain features of the collected different physiological information data, finally combined with the electrical physiological signal and the mechanical physiological signal to re-analyze to obtain the mutual data image of the time-domain features extracted between the electrical physiological signal and the mechanical physiological signal.

Specifically, physiological measurements of heart conditions, blood flow dynamics status, respiration and body movement comprise but is not limited to: body movement, heart rate, heart rate variability, ECG crest composition check, SCG crest composition check, BCG crest composition check, blood pressure, emotion check, etc.

Said data recording unit 2 comprises the central processor for measuring, recording or storing physiological information data collected by said sensor, said central processor is also used for sending said physiological information data to said data analysis unit 3.

Said data analysis unit 3 comprises past database, real-time database and analysis platform that can construct and train algorithm statistical models through machine learning method; said past database comprises: the past physiological information data of said target creature, and the past physiological information data group of the creature of the same or different races or breeds as said target creature; said real-time database comprises the physiological information data of said target creature.

Said data analysis platform is also used for improving the signal-to-noise ratio of said physiological information data, extracting time-domain and/or frequency-domain features of different physiological information data through feature extraction method.

As shown in FIG. 10-12, the structure of the measuring part of the measuring system can be designed to be collapsible, which is convenient for storage and and carrying.

Specifically, the mechanical physiological activity sensor 51 is built into the data collection structure, the data collection structure serves as the carrier of the sensor. The user can directly act on the body of the target creature through the data collection structure to collect physiological information data of the target creature.

For example, collect the body movement data of target creature through accelerometer.

When the invention is applied to the monitoring and analysis of the heart conditions, blood flow dynamics status, respiration and body movement, the collected data are analyzed and give the analysis feedback to the user and/or medical expert. The doctor or other experts then make diagnosis, treatment and prescription recommendation under the guidance of analysis report. This automatic and rapid ECG clinical interpretation and diagnosis greatly improve the profession and efficiency of diagnosis.

Advantageous effect of the invention is: the invention can perform heart health assessment on target creature, for example:

Use heart rate data and blood flow data to judge cardiovascular health and emotion status;

Detect the abnormal heart activities, for example: Arrhythmia;

Detect the blood pressure;

Or use for measurement of lung activities:

Detect respiration rate;

Detect the abnormal respiratory activities;

Or perform measurement of body movement, for example: physical conditions; or judge the overall physical fitness level by respiration data, judge the overall physical fitness level by body movement data, record the real-time and synchronous electrical physiological and mechanical data of heart, respiration and body movement through physiological data collecting platform; the data collected from the sensor will be recorded in the data recording unit 2, the remote end, or storing in other servers or devices.

Above contents are further descriptions of the invention combining with specific preferred embodiments, but cannot limit the embodiment of the invention to these only. For the common technicians who belong to the technical field of the invention, they can make some simple derivations or substitutions on the premise of not departing from the concept of the invention, it should be deemed as belonging to the protection scope of the invention.

1. A hybrid sensing based physiological monitoring and analysis method, wherein comprising the following steps:
S1. Construct algorithm statistical model through experiment;
S2. Collect physiological information data of the target creature; wherein said physiological information data comprises electrical physiological information, mechanical physiological information and body movement data of the target creature;
S3. Perform noise reduction processing on said physiological information data through signal processing method, and extract time-domain features and/or frequency-domain features of different physiological information data through feature extraction method;
S4. Input the time-domain features and/or frequency-domain features extracted from electrical physiological information, mechanical physiological information and body movement data into the algorithm statistical model for calculation, to obtain the output target; wherein, said algorithm statistical model comprises algorithm statistical model of heart rate check, algorithm statistical model of blood pressure check and algorithm statistical model of heart rate variability check, said output target comprises heart rate analysis, blood pressure analysis and heart rate variation analysis corresponding to said algorithm statistical model.
S5. The said output target is used as the analysis report and reported back to the report receiving unit, or said output target is compared with the past database to obtain the analysis report and reported back to the report receiving unit, wherein the said past database comprises: the past physiological information data of said target creature, and the past physiological information data group of the creature of the same or different races or breeds as said target creature.

2. The hybrid sensing based physiological monitoring and analysis method of claim 1, wherein said S1 further comprises the following steps:
S11. Collect the experimental physiological information data of experimental object through the sensor;
S12. Improve signal-to-noise ratio of experimental physiological information data through the signal processing method;
S13. Extract time-domain features and/or frequency-domain features of different experimental physiological information data through the feature extraction method, wherein said feature extraction method is: Fourier Transform, frequency band power calculation, time frequency analysis, wavelet decomposition and waveform detection;
S14. The statistical model is constructed by inputting the time-domain features and/or frequency-domain features of the experimental physiological information data into the machine learning system, and train the statistical model to obtain algorithm statistical model.

3. The hybrid sensing based physiological monitoring and analysis method of claim 2, wherein said S14 further comprises the following steps:
S141. Standard statistical testing parameters and the acceptable deviation degree of algorithm results are preset in said machine learning system;
S142. Said machine learning system extracts the subset of the relevant time-domain features and/or frequency-domain features of said experimental physiological information data through the feature extraction method to construct different combinations of models, and compare the calculation result of the statistical model with the physiological result obtained by the standard measurement method, check whether it meets the preset statistical testing parameter and the acceptable result deviation degree;
S143. If it does not meet, then remove the time-domain features and/or frequency-domain features of the test from said statistical model;
S144. Construct the algorithm statistical model by selecting the feature subset with the highest accuracy and statistical parameter values.

4. The hybrid sensing based physiological monitoring and analysis method of claim 1, wherein said electrical physiological information comprises ECG and electrical respiration measurement diagram.

5. The hybrid sensing based physiological monitoring and analysis method of claim 1, wherein said mechanical physiological information comprises seismocardiogram, ballistocardiogram and mechanical respiration measurement diagram.

6. The physiological detection and analysis method based on hybrid sensing of claim 1, wherein said output target comprises body movement, respiration rate, heart rate, heart rate variability, blood pressure, emotion and cardiac output.

7. A hybrid sensing based physiological monitoring and analysis system, which comprises some sensors, data recording unit, data analysis unit, and report receiving unit; said data analysis unit: used for analyzing the physiological information data of the target creature collected by said sensor after being processed by said data recording unit, and sending the analysis report to said report receiving unit.

8. The hybrid sensing based physiological monitoring and analysis system of claim 7, wherein said sensor comprises electrocardiography sensor, accelerometer, motion sensor and pressure sensor; said data recording unit comprises the central processor for measuring, recording or storing physiological information data collected by said sensor, said central processor is also used for sending said physiological information data to said data analysis unit.

9. The hybrid sensing based physiological monitoring and analysis system of claim 8, wherein said data analysis unit comprises past database, real-time database and analysis platform that can construct and train algorithm statistical models through machine learning method; said past database comprises: the past physiological information data of said target creature, and the past physiological information data group of the creature of the same or different races or breeds as said target creature;

10. The hybrid sensing based physiological monitoring and analysis system of claim 9, wherein said data analysis platform is also used for improving the signal-to-noise ratio of said physiological information data, extracting time-domain features and/or frequency-domain features of different physiological information data through feature extraction method.
